(19) United States
(12) Patent Application Publication
(10) Pub. No.: US 2020/0188297 A1
(43) Pub. Date: Jun. 18, 2020

(54) LXR Agonist in Topical Ophthalmic Formulation for Treatment of Dry-Eye Disorder

(71) Applicant: eyeNOS, Inc., Oak Park, IL (US)

(72) Inventors: Simon Kaja, Yorkville, IL (US); Anita Ghosh, Oak Park, IL (US); Walter Keith Jones, Elgin, IL (US)

(21) Appl. No.: 16/711,646

(22) Filed: Dec. 12, 2019

Related U.S. Application Data
(60) Provisional application No. 62/779,330, filed on Dec. 13, 2018.

(51) Int. Cl. A61K 9/00; A61K 9/51; A61K 31/18; A61P 27/04; A61K 31/195; A61F 9/00

(52) U.S. Cl. CPC A61K 9/0048; A61K 9/5153; A61F 9/0008; A61P 27/04; A61K 31/195; A61K 31/18

(57) ABSTRACT

A topical ophthalmic formulation of an LXR agonist that could be used for treating dry-eye disease. Examples of LXR agonists that could be used include naturally occurring oxysterol compounds, such as ouabagenin or hyodeoxycholic acid, or synthetic, non-sterol compounds such as IMB-151 or T0901317. The LXR agonist compound may be encapsulated within biodegradable nanoparticles, such as poly(lactic/glycolic) acid (PLGA) nanoparticles, micelles, or liposomes. Experimental work establishing proof-of-concept has been performed.

### TECHNICAL FIELD

This invention relates to topical ophthalmic drug therapy for the treatment of dry-eye disorder.

### BACKGROUND

Dry-eye disorder (DED) is a common ocular surface disorder, and one of the most common conditions encountered in ophthalmologic practice. The ocular surface is protected from the external environment by a tear film. This tear film is complex and contains multiple layers secreted by different glands and tissues. The outermost portion of the tear film contains lipids secreted by the Meibomian glands. The middle layer is the aqueous layer containing proteins, electrolytes, and water. The main contributor to this layer is the lacrimal gland, although corneal and conjunctival epithelial cells also contribute. The innermost layer of the tear film is the mucous layer, which contains secreted mucins, electrolytes, and water produced by the conjunctival goblet cells.

Pathophysiologically, DED is a multi-factorial disease manifesting initially with an increase in tear film osmolarity and inflammation of the ocular surface. As the disease progresses, leukocyte infiltration into the lacrimal gland, loss of conjunctival goblet cells, and Meibomian gland dysfunction lead to the commonly observed exacerbation of dry-eye disorder signs and symptoms.

Currently available drug treatments can cause significant instillation pain and various adverse effects. As the need for alternative treatments is apparent, drug agents that can reduce inflammation or protect against the harmful effects of hyperosmolarity could be promising drug candidates for DED. In this regard, the liver X receptors (LXR) are established mediators of lipid-inducible gene expression and are the endogenous receptors for oxysterols. The LXRα subtype has tissue-specific expression, including high expression in macrophages. The LXRβ subtype is ubiquitously expressed.

LXR agonists have effects on lipid metabolism and inflammation. LXR agonists have been shown to exert potent anti-inflammatory effects as negative regulators of macrophage inflammatory gene expression. Specifically, activation of LXR has been demonstrated to improve atherosclerotic lesions in murine models and to modulate inflammatory gene expression in macrophages. Notably, resident macrophages are present in the cornea and conjunctiva, and activation of inflammatory M1 macrophages has been observed in murine DED studies. Thus, the inventors hypothesize that LXR agonists could be effective in treating DED by suppressing ocular inflammation. In addition to having anti-inflammatory effects, LXR agonists also stimulate expression of enzymes involved in lipogenesis. See Steffensen et al, “Putative Metabolic Effects of the Liver X Receptor (LXR)” (2004 February) Diabetes 53 (suppl 1):S36-S42. Since Meibomian gland dysfunction plays a significant role in DED, the inventors also hypothesize that LXR agonists could be effective in treating DED by restoring Meibomian gland function.

### SUMMARY

This invention provides a topical ophthalmic pharmaceutical composition that could be used for treating dry-eye disorder. Dry-eye disorder is a multifactorial disease of the tears and ocular surface that results in symptoms of discomfort, visual disturbance, and tear film instability with potential damage to the ocular surface. It is accompanied by increased osmolarity of the tear film and inflammation. A clinical definition of dry-eye disorder/disease is given in the report of the Tear Film & Ocular Surface Society (TFOS) Dry Eye Workshop (DEWS) II (“TFOS DEWS II Definition and Classification Report”) (2017 July) OculSurf. 15(3):276-283.

In one aspect, the invention is an ophthalmic pharmaceutical composition that comprises a therapeutically effective amount of an LXR agonist compound mixed into an aqueous fluid. The resulting composition may have any liquid mixture form, such as a solution, emulsion, suspension, etc. In some embodiments, the LXR agonist compound is encapsulated within or absorbed onto nanoparticles.

In another aspect, the invention is an ophthalmic pharmaceutical product. The ophthalmic pharmaceutical composition is contained in an eye-dropper container. The product can be a single-use product or a multi-use product. The container may be any suitable type of eye-dropper container, such as bottle, vial, ampoule, etc.

In another aspect, the invention is a method of treating dry-eye disorder comprising administering a therapeutically effective amount of the ophthalmic composition to the patient's eye.

In another aspect, the invention is a method of treating dry-eye disorder by enhancing Meibomian gland lipogenesis or increasing the tear film lipid content, the method comprising administering a therapeutically effective amount of the ophthalmic composition to the patient's eye.

### BRIEF DESCRIPTION OF THE DRAWINGS

FIGS. 1-11 shows the chemical structure of various LXR agonists compounds that could be used in this invention.

FIG. 1 shows hyodeoxycholic acid.

FIG. 2 shows ouabagenin.

FIG. 3 shows ATI-111.

FIG. 4 shows ATI-829.

FIG. 5 shows T0901317.

FIG. 6 shows IMB-151.

FIG. 7 shows IMB-170.

FIG. 8 shows BMS-779788.

FIG. 9 shows BMS-852927.

FIG. 10 shows LXR-623.

FIG. 11 shows GW3965.

FIG. 12 shows a multi-use (multi-dose) eye-dropper vial.

FIG. 13 shows a bundle of multiple single-use vials.

FIGS. 14A and 14B show experimental results relating to tear volumes.

FIGS. 15A and 15B show experimental results relating to corneal fluorescein staining.

FIGS. 16A and 16B show experimental results relating to lacrimal gland histology.

FIGS. 17A and 17B show experimental results relating to conjunctival histology.

FIGS. 18A-18E show experimental results relating to corneal thickness.

FIGS. 19A-19F shows various effects of hyperosmolar stress insult on human corneal epithelial cells.

FIG. 20 shows fluorescence images of ZO-1 at the cell junctions for the HCE-T cells exposed to hyperosmolar stress conditions+/−ouabagenin treatment.

FIG. 21 shows the results of a tear breakup time test in rabbit eyes.

### DETAILED DESCRIPTION

1. Dry-Eye Disorder:

Dry-eye disorder may result from or overlap with other related conditions, including hormone imbalances, various autoimmune diseases (e.g. Sjögren's syndrome, systemic lupus erythematosus, and rheumatoid arthritis), ocular graft-versus-host disease (GVHD), congenital alacrima, xerophthalmia, lacrimal gland ablation, sensory denervation, abnormalities of the lipid tear layer caused by blepharitis or rosacea, abnormalities of the mucin tear layer caused by vitamin A deficiency, trachoma, keratoconjunctivitis, mucocutaneous disorders, aging, menopause, alcohol use disorder, and diabetes.

Other situations that may result in dry-eye disorder include corneal refractive surgery, dry environmental conditions, visual tasking such as computer use, ocular fatigue, contact lens wear, and ocular irritation. Medications which may result in dry-eye disorder include isotretinoin, sedatives, diuretics, tricyclic antidepressants, antihypertensives, oral contraceptives, antihistamines, nasal decongestants, β-blockers, phenothiazines, atropine, and pain-relieving opiates such as morphine.

2. LXR Agonists:

An LXR agonist is a compound that binds and activates the liver X receptors (LXR), which are members of the nuclear receptor superfamily of DNA-binding transcription factors. Any suitable, small-molecule LXR agonist could be used in the ophthalmic composition. In some embodiments, the LXR agonist compound has a molecular weight of less than 650. In some embodiments, the LXR agonist is an oxysterol compound (oxygenated forms of cholesterol or certain bile acids), which can be naturally-occurring or synthetic.

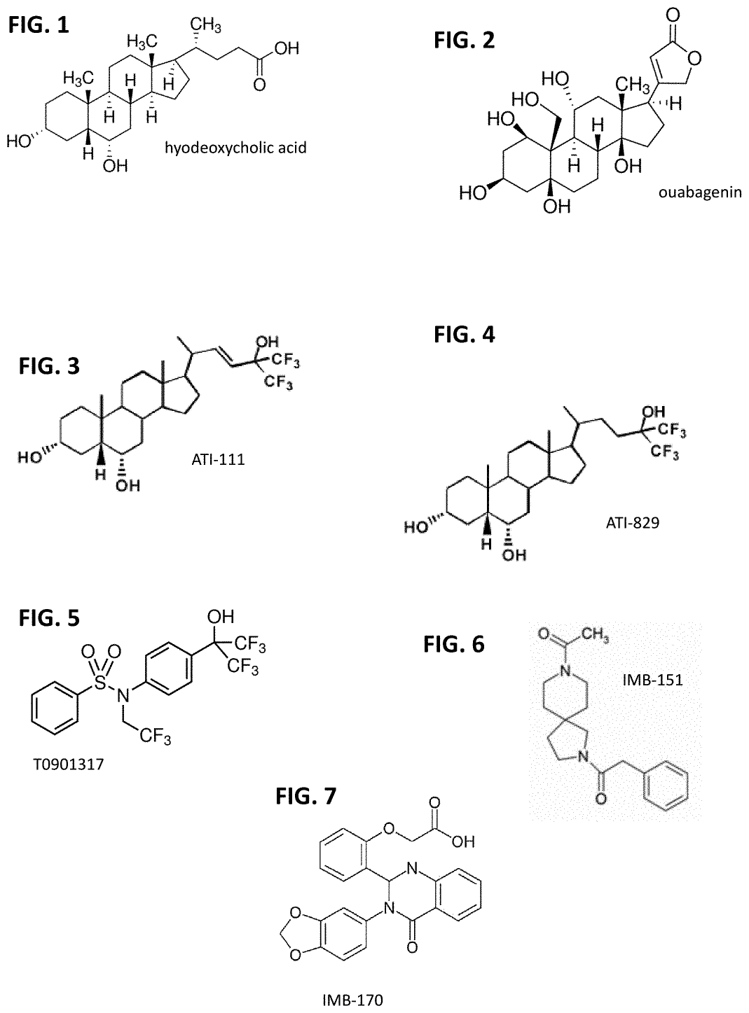

In some embodiments, the LXR agonist is a naturally occurring oxysterol compound. Examples of LXR agonists that are naturally occurring oxysterol compounds include N,N-dimethyl-3β-hydroxy-cholenamide (DMHCA)); 22(R)-hydroxycholesterol; 24(S)-hydroxycholesterol; 22(R)-hydroxycholesterol (220HC); 24(S),25-epoxycholesterol; 24(S)-hydroxycholesterol; 27-hydroxycholesterol (270HC); hyodeoxycholic acid (as shown in FIG. 1); and cholestenoic acid. Another example is ouabagenin as shown in FIG. 2, which is an LXRβ-subtype specific agonist as reported in Tamura et al, “Ouabagenin is a naturally occurring LXR ligand without causing hepatic steatosis as a side effect” (2018 February) Sci Rep. 8: 2305.

In some embodiments, the LXR agonist is a synthetic sterol compound. Examples include ATI-111 and ATI-829 as shown in FIGS. 3 and 4, and as reported in Peng et al, “A novel potent synthetic steroidal liver X receptor agonist lowers plasma cholesterol and triglycerides and reduces atherosclerosis in LDLR^{-/-} mice” (2011 April) Br J Pharmacol. 162(8): 1792-1804. As used herein, the term “sterol compound” means a compound having the well-known steroid core structure of seventeen carbon atoms, bonded in four “fused” rings (six-member rings A, B, and C, and a five member ring D), and with a hydroxyl group at position three. As used herein, the term “synthetic” means that the compound is not naturally occurring in the human body.

In some embodiments, the LXR agonist is a synthetic, non-sterol LXR agonist compound. One example is T0901317 as shown in FIG. 5, which is high potency LXR agonist as described in Houck et al, “T0901317 is a dual LXR/FXR agonist” (2004 August) Molecular Genetics & Metabolism 83:184-187. There is also a related LXR agonist compound T0314407 in which the secondary amine N-(2,2,2-trifluoro-ethyl) of T0901317 is replaced with N-methyl. See Schultz et al, “Role of LXRs in control of lipogenesis” (2000 Nov. 15) Genes Dev. 14(22):2831-2838.

Another example is IMB-151 as shown in FIG. 6, which is an LXRα-specific agonist as reported in Li et al, “Identification of a selective agonist for liver X receptor a (LXRα) via screening of a synthetic compound library” (2014 April) J Biomol Screen. 19(4):566-74. Another example is IMB-170 as shown in FIG. 7, which is an LXRα agonist as reported in Li et al, “Identification of a novel partial agonist of liver X receptor a (LXRα) via screening” (2014) Biochemical Pharmacology 92:438-447.

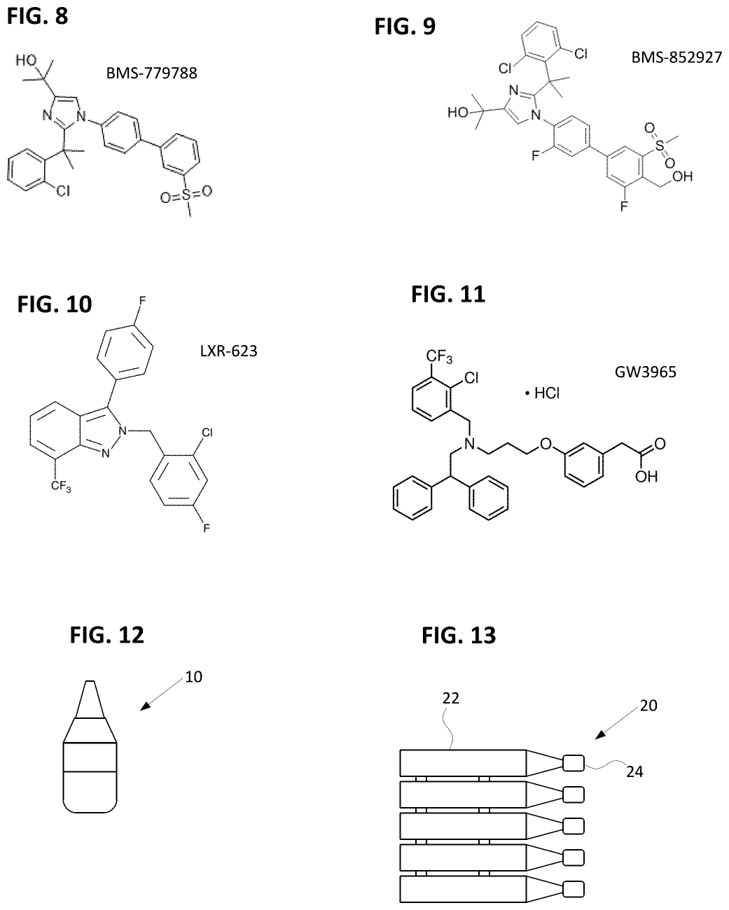

Another example is BMS-779788 (CAS No. 918348-67-1) as shown in FIG. 8 and reported in Kirchgessner et al, “Pharmacological Characterization of a Novel Liver X Receptor Agonist with Partial LXRα Activity and a Favorable Window in Nonhuman Primates” (2015 February) Journal of Pharmacology & Experimental Therapeutics 352(2):305-314. Another example is BMS-852927 (CAS No. 1256918-39-4) as shown in FIG. 9 and reported in Kirchgessner et al, “Beneficial and Adverse Effects of an LXR Agonist on Human Lipid and Lipoprotein Metabolism and Circulating Neutrophils” (2016 August) Cell Metab. 24(2):223-33.

Another example is R211945 as reported by Vucic et al, “Regression of inflammation in atherosclerosis by the LXR agonist R211945: a noninvasive assessment and comparison with atorvastatin” (2012 August) JACC Cardiovasc Imaging 5(8):819-28. Another example is LXR-623 (CAS No. 875787-07-8) as shown in FIG. 10 and reported in Katz et al, “Safety, pharmacokinetics, and pharmacodynamics of single doses of LXR-623, a novel liver X-receptor agonist, in healthy participants” (2009 June) J Clin Pharmacol. 49(6):643-9.

Another example is GW3965 as shown in FIG. 11 and reported in Peng et al, “A novel potent synthetic steroidal liver X receptor agonist lowers plasma cholesterol and triglycerides and reduces atherosclerosis in LDLR^{-/-} mice” (2011 April) Br J Pharmacol. 162(8):1792-1804. Other LXR agonists that could be used are disclosed in Ma et al, “Liver X Receptors and their Agonists: Targeting for Cholesterol Homeostasis and Cardiovascular Diseases” (2017) Curr. Issues Mol. Biol. 22:41-64, which is incorporated by reference herein.

The LXR agonist may be a full or partial agonist. The LXR agonist may have any suitable potency for therapeutic effectiveness in treating dry-eye disorder. In some embodiments, the LXR agonist has an in-vitro potency greater than EC₅₀ value of 5 μM for LXRα, LXRβ, or both; in some cases, potency greater than 1 μM; in some cases, potency greater than 500 nM; and in some cases, potency greater than 100 nM.

The LXR agonist may be subtype-specific. In some embodiments, the LXR agonist is selective for the LXRα subtype (i.e. having greater potency for the α-subtype than the β-subtype), such as T0901317 or IMB-151. In some embodiments, the LXR agonist has a potency for LXRα that is at least 5-fold greater than for LXRβ; in some cases, at least 10-fold; and in some cases, at least about 50-fold.

In some embodiments, the LXR agonist is selective for the LXRβ subtype (i.e. having greater potency for the β-subtype than the α-subtype), such as ouabagenin. In some embodiments, the LXR agonist has a potency for LXRβ that is at least 5-fold greater than for LXRα; in some cases, at least 10-fold; and in some cases, at least about 50-fold.

Any therapeutically effective amount of the LXR agonist compound may be used in the topical ophthalmic composition. In some embodiments, the concentration of the LXR agonist compound in the topical ophthalmic composition is 10 mg/ml or lower; in some cases, 5 mg/ml or lower; in some cases, 3 mg/ml or lower; and in some cases, 1 mg/ml or lower. In some embodiments, the concentration of the LXR agonist compound in the topical ophthalmic composition is in the range of 0.25 mg/ml to 10 mg/ml; in some cases, in the range of 0.25 mg/ml to 5 mg/ml; in some cases, in the range of 0.25 mg/ml to 3 mg/ml; and in some cases, in the range of 0.25 mg/ml to 1 mg/ml. These effective concentrations are supported by the experimental data, with higher amounts needed in the clinical treatment context after factoring in release kinetics, loss in administration, dilution or wash-away by tear fluid, etc.

In some embodiments, the concentration of the LXR agonist compound in the topical ophthalmic composition is 100 mg/ml or lower; in some cases, 50 mg/ml or lower; in some cases, 25 mg/ml or lower; and in some cases, 10 mg/ml or lower. In some embodiments, the concentration of the LXR agonist compound in the topical ophthalmic composition is in the range of 1 mg/ml to 100 mg/ml; in some cases, in the range of 1 mg/ml to 50 mg/ml; in some cases, in the range of 1 mg/ml to 25 mg/ml; and in some cases, in the range of 1 mg/ml to 10 mg/ml. These effective concentrations are supported by the experimental data, with higher amounts needed in the clinical treatment context after factoring in release kinetics, loss in administration, dilution or wash-away by tear fluid, etc.

3. Other Formulation Ingredients & Properties:

As used herein, “topical ophthalmic” means any pharmaceutical formulation that is applied directly to the ocular surface, such as the front of the eye (e.g. on the cornea), under the upper eyelid, on the lower eyelid and in the cul-de-sac, etc. The topical ophthalmic composition is a liquid composition that comprises an aqueous fluid. As used herein, the term “aqueous fluid” means a fluid that is at least 75% water by weight; and in some cases, at least 90%. The liquid composition may have the form of any of the various types of liquid mixtures, such as a solution, suspension, emulsion, gel, sol, liquid foam, etc.

In some embodiments, the ophthalmic compositions may further comprise other ingredients, such as surfactants, adjuvants, buffers, antioxidants, tonicity adjusters, preservatives (e.g. EDTA, benzalkonium chloride), sodium chlorite, sodium perborate, polyquaterium-1), thickeners, or viscosity modifiers. Examples of thickeners or viscosity modifiers that could be used include carboxymethyl cellulose, hydroxymethyl cellulose, polyvinyl alcohol, polyethylene glycol, glycol 400, propylene glycol hydroxymethyl cellulose, hyaluronic acid, hydroxypropyl methylcellulose, and the like.

The pH of the ophthalmic composition may be in any suitable range, such as pH 5.0 to 8.5. The pH of the ophthalmic fluid composition may be adjusted by adding any physiologically and ophthalmically acceptable pH-adjusting acids, bases, or buffers to a suitable range. The ophthalmic compositions of the invention can further contain pharmaceutical excipients suitable for the preparation of ophthalmic formulations. Examples of such excipients are preserving agents, buffering agents, chelating agents, antioxidant agents and salts for regulating the osmotic pressure.

4. Nanoparticle Formulation:

Nanotechnology-based ocular drug delivery platforms could increase drug bioavailability to the eye. In particular, because a nanoparticle formulation will tend to accumulate in the conjunctival cul-de-sac, the contact time of the nanoparticle drug is considerably longer than comparable ophthalmic solutions. This increased contact time may give a longer duration of action.

In some embodiments, the LXR agonist compound is adsorbed onto or entrapped within nanoparticles. The term “nanoparticle” encompasses particles, nanospheres, nanocapsules, liposomes, polymeric micelles, quantum dots, dendrimers, solid lipid nanoparticles, etc. Examples of nanoparticle formulations that could be used for ocular drug delivery are described in Deepak Thassu & Gerald Chader (eds.), Ocular Drug Delivery Systems: Barriers and Application of Nanoparticulate Systems (2013) CRC Press and Kewal K. Jai n, “Nanocarriers for Ocular Drug Delivery” in The Handbook of Nanomedicine (2008) Humana Press.

The nanoparticles may be made of any suitable material, including biocompatible polymers or biologic materials. Examples of such materials include chitosan, a polycarboxylic acid such as polyacrylic acid, hyaluronic acid esters, polyitaconic acid, poly(butyl)cyanoacrylate, poly-ε-caprolactone, poly(isobutyl)caprolactone, poly(lactic/glycolic) acid, or poly(lactic acid), poly(ethylene glycol)-block-poly(L-lysine), EUDRAGIT® RS100, or EUDRAGIT® RL100. The EUDRAGIT® materials are copolymers of ethyl acrylate, methyl methacrylate, and a low content of methacrylic acid ester with quaternary ammonium groups.

In some embodiments, the nanoparticles comprise polymers that are both biocompatible and biodegradable, such as polylactic acid, polyglycolic acid, poly(lactic/glycolic) acid, poly(caprolactone), polyhydroxobutyrate, chitosan, hyaluronic acid, poly(2-hydroxyethyl-methacrylate), and poly(ethylene glycol). In some cases, the nanoparticles comprise synthetically-made polymers that are both biocompatible and biodegradable.

The nanoparticles may have any suitable size less than 1 μm. In some embodiments, the nanoparticles have an average size diameter in the range of 100-700 nm. The resulting nanoparticle formulation may be in any suitable liquid form, including suspension, emulsion, gel, sol, liquid foam, etc.

5. Packaging:

The ophthalmic composition may be packaged in any suitable manner, such as a ready-to-use single-use product or a multi-use product. The single-use product is intended to be consumed in a single setting. The ophthalmic composition may be provided in any suitable type of eye-dropper container for topical ocular administration. In some embodiments, the product is a multi-use product and the eye-dropper container contains 3-20 mL volume of the ophthalmic composition. As an example, FIG. 12 shows a multi-use (multi-dose) eye-dropper vial 10 containing the ophthalmic composition. In some embodiments, the product is a single-use product and the eye-dropper container contains less than 1 mL volume of the ophthalmic composition. As an example, FIG. 13 shows a bundle 20 of multiple single-use vials 22 containing the ophthalmic composition. For administering a dosage, a vial 22 is detached from the bundle 20 and cap 24 is twisted off to open the vial tip.

6. Method of Treatment:

The ophthalmic composition of the invention may be used for treating dry-eye disorder. As used herein, the term “treating” also encompasses preventing dry-eye disorder. One or more particular symptoms of dry-eye disorder could be ameliorated, including eye discomfort, visual disturbance, tear film instability, tear hyperosmolarity, and inflammation of the ocular surface.

The patient receives the ophthalmic composition topically by instilling droplets directly onto the eye surface (which encompasses the conjunctival sac), for one or both eyes. Topical administration of the ophthalmic composition may be performed by the patient themselves. Or it could be performed by someone else, such as a caregiver, a spouse, a clinician (e.g. physician or nurse), etc.

The patient may receive intermittent dosing of the ophthalmic composition. In some embodiments, the topical ophthalmic composition is administered once daily. In some embodiments, the topical ophthalmic composition is administered twice daily. In some embodiments, the topical ophthalmic composition is administered three times daily.

The anti-inflammatory properties of LXR agonists may have a role in the therapeutic effectiveness. However, the ocular environment possesses special immune privilege that already protects it from inflammatory insults. See Zhou et al, “Ocular immune privilege” (2010) F1000 Biology Reports 2:3. This ocular immune privilege is maintained by multiple mechanisms, including physical barriers (by an efficient blood-retina barrier and lack of efferent lymphatics) that obstruct cell migration and diffusion of large molecules into and out of the eye; presence of inhibitory soluble and cell-bound factors that create an immunosuppressive microenvironment; and active regulation of the systemic immune response by the eye. As a result, systemic anti-inflammatory responses are very different from ocular inflammatory responses. Thus, the inventors hypothesize that the therapeutic effectiveness of LXR agonists in dry-eye disorder could also be the result of stimulating lipogenesis, for example, in the Meibomian glands. This lipogenic mechanism of action may work in parallel or independent of the anti-inflammatory action.

### EXPERIMENTAL EXAMPLES

1. Preparation of PLGA-Nanoparticles:

Nanoparticle-encapsulated drug was prepared for animal experiments. Test compound T0901317 was encapsulated in poly(lactic/glycolic) acid (PLGA) nanoparticles using a variation of the oil-in-water single emulsion technique reported by Langert et al, “Attenuation of experimental autoimmune neuritis with locally administered lovastatin-encapsulating poly(lactic-co-glycolic) acid nanoparticles” (January 2017) J Neurochem. 140(2):334-346. A suitable amount of the T0901317 drug was used to result in a drug loading of about 25%. The T0901317 drug was dissolved in 1 mL of dichloromethane. 100 mg of ester-terminated PLGA (85:15) was dissolved in the dichloromethane and slowly added to ice-cold 1% (w/v) polyvinyl alcohol (10 mL) while vigorously mixing. The polyvinyl alcohol serves as a surfactant and facilitates production of nanoparticle by preventing aggregation. The resultant suspension was emulsified by probe sonication and then diluted with 100 mL ice-cold polyvinyl alcohol. The organic solvent was evaporated by constant stirring for 3 hours at 25° C. The resulting PLGA nanoparticles were isolated by centrifugation (25,000 g for 25 min at 4° C.) and washed with deionized water. The release profile of T0901317 drug was typical for PLGA nanoparticle-drug formulations. The PLGA particle diameter was 800±50 nm, the calculated drug loading was about 23%, and the Zeta potential was −30.00±5.00 mV. A drug loading of 25% converts to about 3.3 mg/ml or 7 mM concentration of T0901317.

2. Mice Experiments:

The effect of PLGA-T0901317 was tested in a preclinical mouse model for DED, as compared to empty PLGA nanoparticles (vehicle control). Both vehicle and drug nanoparticles were made as preservative-free ophthalmic suspensions. The mice experiments were performed in a manner similar to that described in Žiniauskaitė et al, “Efficacy of Trabodenoson in a Mouse Keratoconjunctivitis Sicca (KCS) Model for Dry-Eye Syndrome” (June 2018) Investigative Ophthalmology & Visual Science 59:3088-3093. In mice 6-10 weeks age, experimental DED was induced by scopolamine administration in a desiccating environment. Scopolamine was administered by placing a 3×3 mm piece of a scopolamine patch into each ear. Proper placement of the patches was checked daily and the patches were replaced twice weekly.

Concomitantly, mice were placed in a controlled desiccating environment of 5-15% humidity and 15 L/min airflow for 14 days. Air was dried using an in-line water separator and two in-line custom built 4 L-capacity desiccating columns filled with orange silicagel desiccant. The dried air was distributed via a four-channel manifold into four individual flow meters, which were used to regulate airflow to 15 L/min into each of four cages per system. The dried air was pumped into each cage through two access points that were placed 15 cm apart and 4 cm above floor level, which corresponds to the height of the mouse's eyes.

The ophthalmic suspensions were administered to both eyes three times daily by pipetting 5 μL into the conjunctival sac using a P20 micropipettor. Treatments were started 3 days before induction of experimental DED and continued throughout the duration of the 2-week induction period. At the end of the study, mice were euthanized by overexposure to CO₂ followed by cardiac puncture. The eyes, including the lids and lacrimal glands, were dissected and post-fixed overnight in 4% paraformaldehyde.

(2a) Ocular Surface Inflammation (Live Animals):

Ocular surface inflammation was measured by corneal fluorescein staining. 1 μL of 0.05% liquid sodium fluorescein were applied into the conjunctival sac of both eyes. After 90 seconds, corneal epithelial damage was assessed by imaging with a fluorescence microscope. The total corneal fluorescein score was calculated by assessing fluorescein puncta and patches per a scoring system: absent, 0; slightly punctate staining, 1; strong punctate staining but not diffuse, 2; small positive plaque areas, 3; large area fluorescein plaque, 4.

(2b) Histological Analysis:

Ocular tissue was serially sectioned at 10 μm thickness. A series of sections were selected and processed for routine hematoxylin/eosin staining for lacrimal gland and corneal epithelium morphology or periodic acid-Schiff (PAS) staining for visualization of goblet cells. The stained sections were imaged under a light microscope. Histological analysis of the lacrimal glands were performed to identify lymphocytes. Histological grading of inflammatory lesions was performed as follows: 1 for 1-5 foci composed of >20 mononuclear cells per focus; 2 for >5 such foci, but without significant parenchymal destruction; 3 for degeneration of parenchymal tissue; 4 for extensive infiltration of the glands with mononuclear cells and extensive parenchymal destruction; and 5 for severe destructive foci with focal fibrosis, ductal dilation, and/or fatty infiltration in addition to grade 4 lesions. Histological analysis of the corneal sections were performed to measure corneal tissue thickness. Thickness of the epithelial cell layer, the number of epithelial cells in the layer, the stroma and total corneal thickness were quantified. Mean thickness was determined by averaging five separate measurements for each corneal section. Histological analysis of the goblet cells in the conjunctival fornix were performed to count goblet cells. Goblet cells quantification is expressed as number of goblet cells per mm of conjunctival length.

(2c) Quantification of Tear Volume:

Tear volume quantification was performed using a sterile phenol red-soaked cotton thread that was applied in the lateral canthus for a duration of 10 s, using forceps. The wetting length of the thread was read under a microscope and estimated using a ruler. Resolution of the measurements was 0.5 mm. Tear volume was measured in all groups, at baseline, and on study days 4, 7, 11, and 14.

(2d) Results:

Parametric data was analyzed using Student's t test, while non-parametric data was analyzed using Mann-Whitney ranks test. Data are presented as mean±S.D. or s.e.m., or as median±interquartile range. Differences were considered statistically significant at P<0.05. On visual inspection of the eyes, there was no evidence of hyperemia or ocular irritation to suggest any ocular toxicity or adverse effects.

Body Weights.

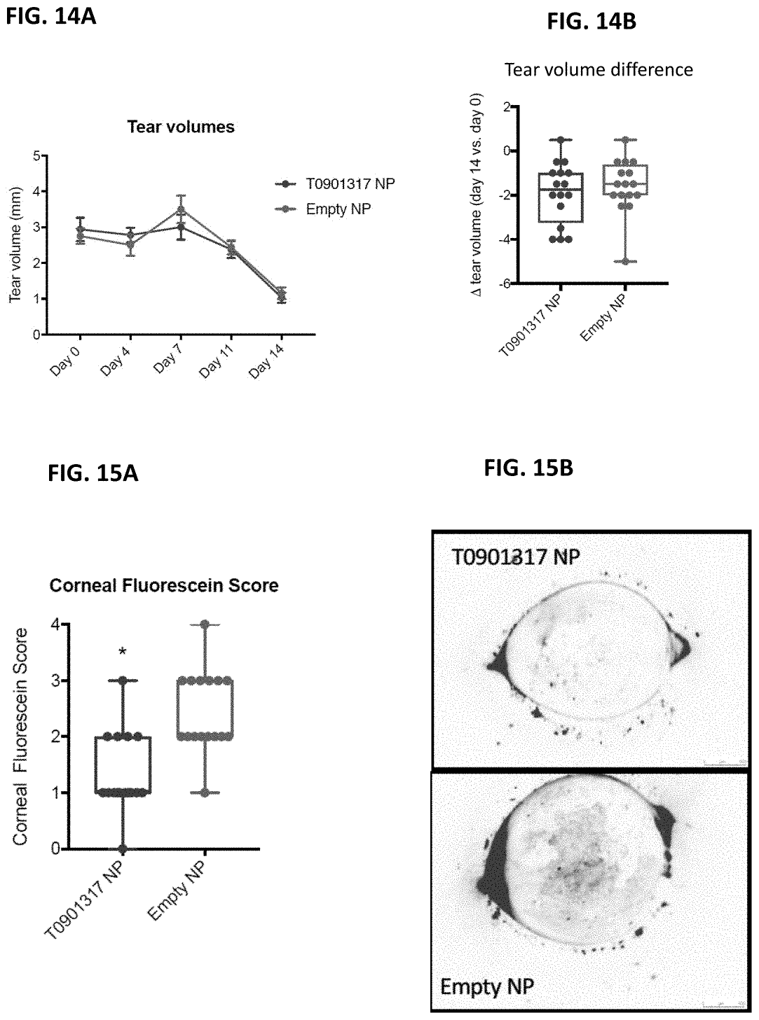

There was a statistically significant reduction in body weights following induction of dry-eye disease for 2 weeks (P<0.001), typical for DED mice model induced by the SiccaSystem™ technique. However, there were no differences in the extent of body weight loss between treatment groups (P=0.23). Tear volumes. Successful DED induction was confirmed by reduced tear volumes after 14 days of exposure to the desiccating environment with scopolamine administration. As shown in FIG. 14A, tear volumes were reduced from 2.9±0.5 mm to 1.0±0.2 mm (p<0.001) in the PLGA-T0901317 group. And from 2.8±0.3 mm to 1.2±0.2 mm (p<0.001) in the PLGA-empty group. As shown in FIG. 14B, there was no statistically significant difference in the extent of tear volume reduction between the groups (p=0.50).

Corneal Fluorescein Staining.

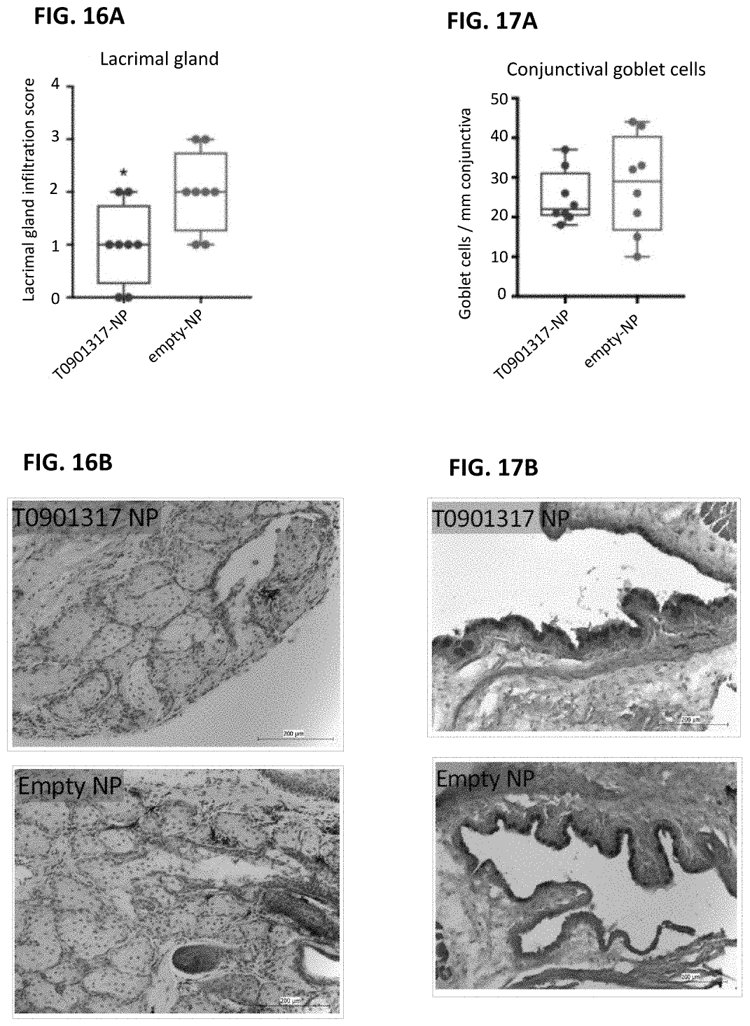

PLGA-encapsulated T0901317 reduced corneal fluorescein staining in the mice DED model, suggestive of anti-inflammatory effects. As shown in FIG. 15A, the two weeks of PLGA-T0901317 treatment caused a statistically significant reduction in corneal fluorescein staining score (p<0.05) compared with empty nanoparticles, indicating suppression of corneal surface inflammation. Representative images are shown in FIG. 15B. Lacrimal gland histology. Similarly, as shown in FIG. 16A, analysis of lacrimal gland pathology (scored according to the scale given above) demonstrated significantly reduced infiltration of leukocytes for PLGA-T0901317-treated eyes compared with eyes treated with empty PLGA nanoparticles (p<0.05). FIG. 16B shows representative images of stained lacrimal gland sections. The top panel is from a PLGA-T0901317 treated eye. The bottom panel is from a PLGA-empty treated eye. Conjunctival histology. FIG. 17A shows the number of goblet cells counted in PAS-stained sections of the conjunctiva. There was no statistically significant difference between PLGA-T0901317 and PLGA-empty treated eyes (p=0.54). FIG. 17B shows representative images of PAS-stained sections of the conjunctiva. The top panel is from a PLGA-T0901317 treated eye. The bottom panel is from a PLGA-empty treated eye.

Corneal Thickness.

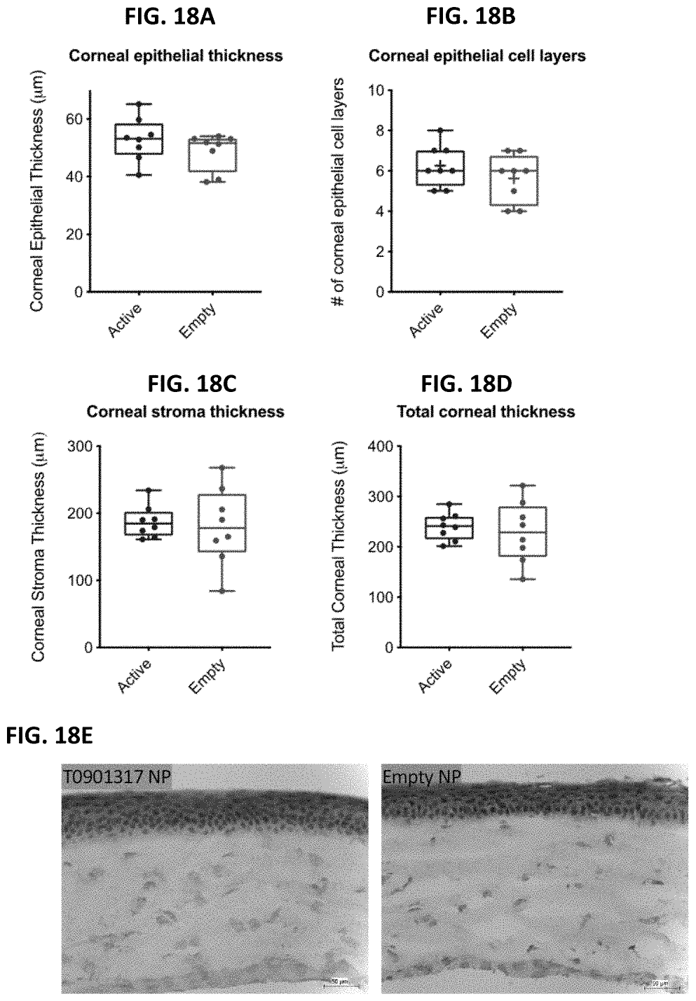

FIGS. 18A-18D shows corneal thickness data as box and whisker plots that show means, interquartile ranges, and min/max values for each treatment group. FIG. 18A shows the corneal epithelial thickness (p=0.25). FIG. 18B shows the number of cells in the epithelial cell layer (p=0.40). FIG. 18C shows the corneal stroma thickness (p=0.76). FIG. 18D shows the total corneal thickness (p=0.64). There were no statistically significant differences between the treatment groups. This confirms the above-mentioned visual observations that there was no evidence that the drug treatment has ocular toxicity or adverse effects.

3. In-Vitro Experiments:

The anti-inflammatory effects of T0901317 were studied on in-vitro cell culture of human corneal epithelial cells (HCE-T). HCE-T cells were grown in DMEM/F12 (1:1), 5% fetal bovine serum, penicillin-streptomycin, 5 mg/ml insulin, 10 ng/ml human recombinant epidermal growth factor and 0.5% dimethyl sulfoxide in 5% CO₂ at 37° C.

(3a) Hyperosmolar Stress Insult:

To simulate dry-eye conditions, the cells were exposed to hyperosmolar stress insult as described in Chen et al, “Hyperosmolarity-Induced Cornification of Human Corneal Epithelial Cells Is Regulated by JNK MAPK” (February 2008) Investigative Ophthalmology & Visual Science, 49(2):539-549. Confluent HCE-T were passaged into 12-well plates containing KSFM media (keratinocyte-SFM media) for 24 hours, and then NaCl was added to wells in concentrations to produce media osmolarities in the range of 350-800 mOsm.

(3b) MTT Cell Viability Assay:

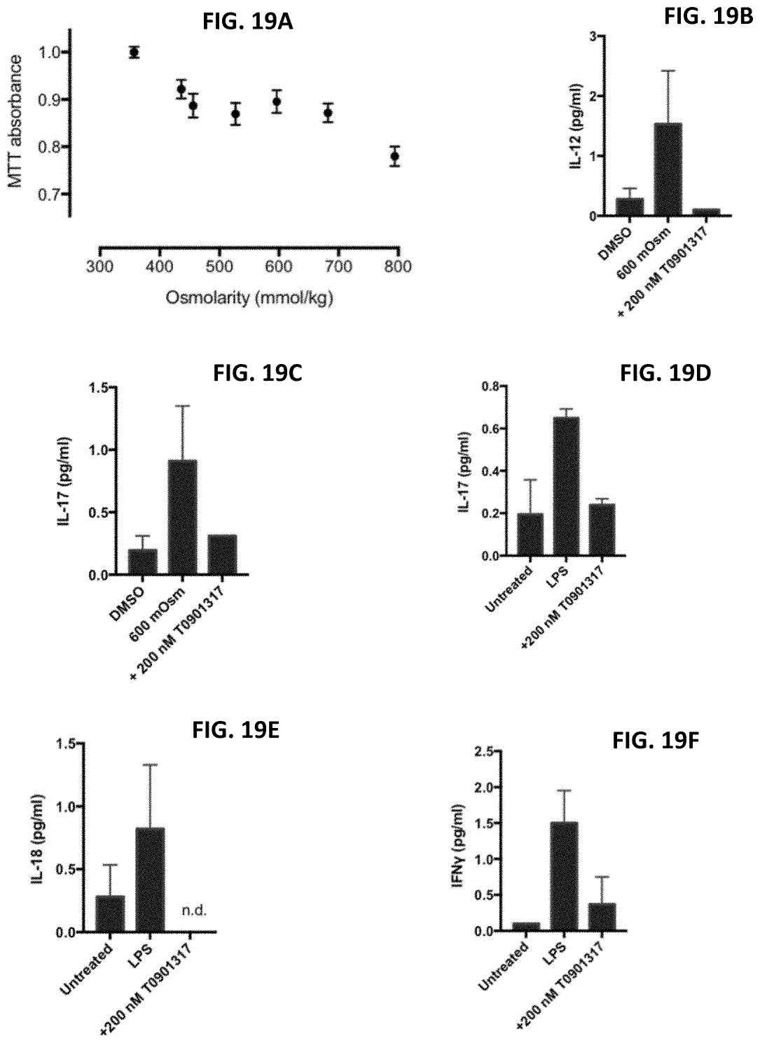

MTT cell viability assay was performed on the hyperosmolar-stressed cells by MTT absorbance measured at 570 nm wavelength. The relative fluorescence units (RFU) of background was measured and deducted. Cell viability (% of control) was calculated as (RFU_{treated}/RFU_{control})×100. FIG. 19A shows the effect of hyperosmolar stress on the HCE-T cells. Cell viability was around 85-90% within the clinically relevant range of hyperosmolar conditions (350-700 mOsm) and only decreased further under conditions in excess of 700 mOsm.

(3c) Cytokines:

Multiplex cytokine expression analysis was performed to determine the anti-inflammatory effects of T0901317 (at 200 nM). Consistent with previous reports, FIGS. 19B and 19C shows that hyperosmolar stress of 600 mOsm for 20 hours increased secretion of both IL-12 and IL-17. But preincubation with the T0901317 compound (200 nM, for 1 hour) fully suppressed the hyperosmolar stress-induced secretion of IL-12 and IL-17.

Lipopolysaccharide (LPS)-induced (10 ng/ml) secretion of IL-17, IL-18, and IFNγ was also examined. Similar to the results with hyperosmolar stress, preincubation with the T0901317 compound suppressed LPS-induced secretion of IL-17 (see FIG. 19D), IL-18 (see FIG. 19E), and IFNγ (see FIG. 19F). These cytokine expression results demonstrate the potent anti-inflammatory effects of LXR activation. Notably, the concentration of T0901317 used was significantly below the EC₅₀ for activation of the retinoid X receptor (5 μM). Topical application is not expected to result in activation of the pregnane X receptor (PXR), supporting the notion that the anti-inflammatory effects observed here, both in-vivo and in-vitro, can be attributed to LXR activation.

(3d) HCE-T Cells:

The corneal epithelium serves as a physiological barrier. However, inflammation and the ensuing oxidative stress can impair barrier function and contribute to the exacerbation of inflammatory processes and increase susceptibility to other ocular surface conditions, such as corneal keratitis. The effect of ouabagenin on the barrier function of human corneal epithelial (HCE-T) cells was tested in in-vitro experiments. The cells were seeded at 25,000 cells/well in 8-chamber slides. Cells were incubated for 3 days at 37° C. until confluent. Cells were pre-treated with DMSO (vehicle) or ouabagenin (0.05 μM in DMSO). One hour later, cells received treatment of 0 (media), 25, 50, or 75 mM NaCl to mimic hyperosmolar conditions. Cells were incubated overnight at 37° C.

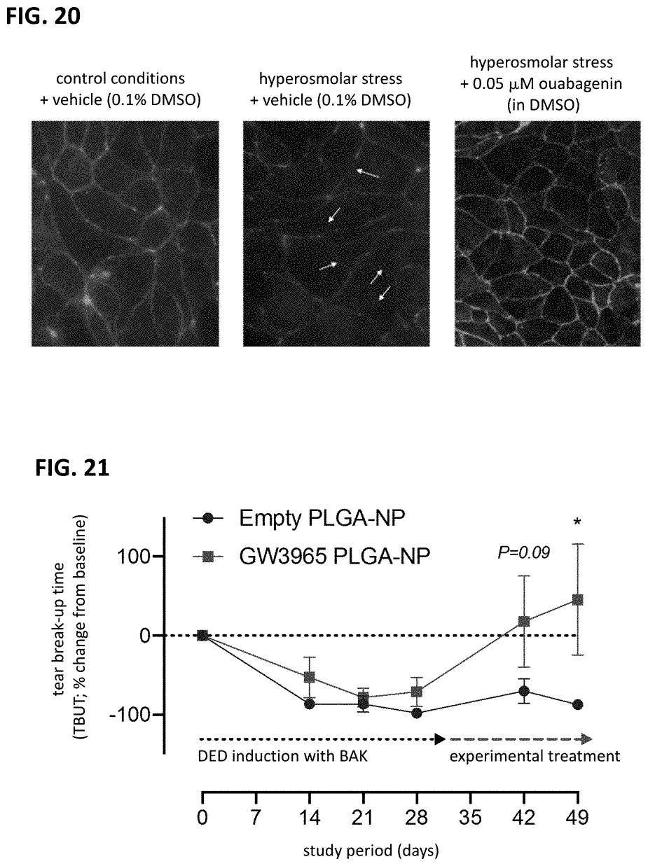

Zona occludens 1 (ZO-1) is a tight junction protein that is involved in the barrier function of corneal epithelium. The presence of ZO-1 (or absence thereof) is an indicator of the health of this barrier. In this experiment, immunocytochemistry for ZO-1 was performed on the HCE-T cells after the overnight incubation. Slides were imaged under a fluorescent microscope. FIG. 20 shows representative fluorescence images of ZO-1 at the cell junctions for the HCE-T cells exposed to 50 mM NaCl hyperosmolar stress conditions. As seen here, exposure of the HCE-T cells to hyperosmolar stress conditions resulted in loss of ZO-1, with the cell barrier lines being compromised at multiple locations (see middle panel, indicated by white arrows). However, treatment with ouabagenin protected against this loss of ZO-1 barrier function (see right panel).

4. Rabbit Experiments:

The effect of GW3965 was tested in a preclinical rabbit model for DED. New Zealand White rabbits (Charles Rivers, France) of age five-to-seven months were used for the rabbit experiments. The rabbits were housed at a constant temperature (22±1° C.), at humidity of 55%±10%, and in a light-controlled environment (lights on from 7 am to 7 μm) with ad libitum access to food and water. The rabbits were induced into DED ocular surface pathology by benzalkonium chloride (BAK; Sigma-Aldrich, St. Louis, Mo.). BAK was made into 0.1% saline solution, which was applied topically to the rabbit eyes (60 μl per eye) twice daily bilaterally for 35 days. This BAK-induced ocular surface pathology to mimic DED is described in Xiong et al. (2008) Investigative Ophthalmology & Visual Science, vol. 49(5):1850-6

The test agent, GW3965, was encapsulated in PLGA-nanoparticles (at about 1 μM concentration of GW3965, or about 26 μg/ml) was prepared in the same manner as explained above. After 35 days to induce DED ocular surface pathology, BAK administration was discontinued and treatment with PLGA-GW3965 was started. The PLGA-GW3965 test agent was administered bilaterally once daily (25 μl/eye) through day 49.

Tear breakup time (TBUT) test was used to quantify ocular surface pathology and was performed on days 0, 14, 21, 28, 42, and 49 using a fluorescein sodium ophthalmic strip. After a blink, the tear film was observed under a slit lamp using broad cobalt blue illumination. TBUT was recorded as the number of seconds that elapsed between the last blink and the appearance of the first dry spot on the tear film. Data were analyzed and expressed as % change from baseline (day 0) for each rabbit.

4(a) Results:

Data are from n=3 rabbits (n=6 eyes per group) and are presented as mean±s.e.m. in FIG. 21, which shows the progression of TBUT. As expected for BAK-induced ocular surface pathology, TBUT progressively decreased with ongoing BAK treatment. Upon discontinuation of BAK, TBUT in the treatment group (GW3965-PLGA) were restored to values similar to baseline. In contrast, TBUT in the control group (empty PLGA-nanoparticles) remained impaired, indicating chronic ocular surface pathology. Data were analyzed using mixed effects model, with Sidak multiple comparisons test (day 42: P=0.09; day 49: P<0.05).

The foregoing description and examples have been set forth merely to illustrate the invention and are not intended to be limiting. Each of the disclosed aspects and embodiments of the invention may be considered individually or in combination with other aspects, embodiments, and variations of the invention. In addition, unless otherwise specified, the steps of the methods of the invention are not confined to any particular order of performance. Modifications of the disclosed embodiments incorporating the spirit and substance of the invention may occur to persons skilled in the art, and such modifications are within the scope of the invention.

Any use of the word “or” herein is intended to be inclusive and is equivalent to the expression “and/or,” unless the context clearly dictates otherwise. As such, for example, the expression “A or B” means A, or B, or both A and B. Similarly, for example, the expression “A, B, or C” means A, or B, or C, or any combination thereof.

1. A method of treating dry-eye disorder in a patient, comprising:
administering an ophthalmic pharmaceutical composition topically onto the patient's eye;
wherein the ophthalmic pharmaceutical composition comprises an aqueous fluid and an LXR agonist compound.

2. The method of claim 1, wherein the LXR agonist compound is encapsulated within or absorbed onto nanoparticles.

3. The method of claim 2, wherein the nanoparticles comprise poly(lactic/glycolic) acid.

4. The method of claim 3, wherein the nanoparticles have an average size diameter in the range of 100-700 nm.

5. The method of claim 1, wherein the ophthalmic pharmaceutical composition is administered twice or three times daily.

6. The method of claim 1, wherein the ophthalmic pharmaceutical composition is administered once daily.

7. The method of claim 1, wherein the LXR agonist compound is LXRα subtype specific.

8. The method of claim 1, wherein the LXR agonist compound is LXRβ subtype specific.

9. The method of claim 1, wherein the concentration of the LXR agonist compound in the topical ophthalmic composition is 10 mg/ml or less.

10. The method of claim 9, wherein the concentration of the LXR agonist compound in the topical ophthalmic composition is 0.25 mg/ml-10 mg/ml.

11. The method of claim 1, wherein the concentration of the LXR agonist compound in the topical ophthalmic composition is less than 100 mg/ml.

12. The method of claim 1, wherein the LXR agonist compound is a synthetic, non-sterol compound.

13. The method of claim 1, wherein the LXR agonist compound is a sterol compound.

14. The method of claim 1, wherein the ophthalmic pharmaceutical composition is provided in a single-use eye-drop container containing less than 1 mL of the topical ophthalmic composition.

15. The method of claim 1, wherein the ophthalmic pharmaceutical composition is provided in a multi-use eye-drop container containing 3-20 mL volume of the topical ophthalmic composition.

16. An ophthalmic pharmaceutical composition, comprising:
nanoparticles comprising a biocompatible polymer;
an LXR agonist compound encapsulated within or absorbed onto the nanoparticles;
an aqueous fluid into which the nanoparticles and LXR agonist compound are mixed.

17. The pharmaceutical composition of claim 16, wherein the concentration of the LXR agonist compound is 10 mg/ml or less.

18. The pharmaceutical composition of claim 16, wherein the nanoparticles have an average size diameter in the range of 100-700 nm.

19. An ophthalmic pharmaceutical product, the product comprising:
an eye-dropper container;
an ophthalmic pharmaceutical composition contained in the container, the ophthalmic composition comprising:
nanoparticles comprising a biocompatible polymer;
an LXR agonist encapsulated within or absorbed onto the nanoparticles;
an aqueous fluid into which the nanoparticles and LXR agonist compound are mixed.

20. The pharmaceutical product of claim 19, wherein the product is a single-use product and the eye-dropper container contains less than 1 mL of the ophthalmic composition; or wherein the product is a multi-use product and the eye-dropper container contains 3-20 mL volume of the ophthalmic composition.
